(19) United States
(12) Patent Application Publication
(10) Pub. No.: US 2006/0173416 A1
(43) Pub. Date: Aug. 3, 2006

(54) Syringe with coordinated inserts

(76) Inventor: Christopher S. Young, South Kent, CT (US)

Correspondence Address: Arthur G. Schaier;Carmody & Torrance LLP, 50 Leavenworth Street, P.O. Box 1110, Waterbury, CT, 06721-1110, US

(21) Appl. No.: 10/983,471

(22) Filed: Nov. 8, 2004

(51) Int. Cl. A61M 5/00

(52) U.S. Cl. 604/187

(57) ABSTRACT

A syringe for at least one of drawing a fluid into the syringe and dispensing a fluid substance therefrom, the syringe comprising a barrel comprising an inner surface defining a cylindrical chamber for retaining a dispensable fluid therein, a distal end terminating in a tip to which a needle is coupleable, and an open proximal end; an elongated sleeve positionable within the cylindrical chamber, the sleeve having an outer diameter dimensioned to be friction-fittable in the cylindrical chamber; a plunger having a distal end and a proximal end, at least one of which is capped, the plunger being dimensioned to be slideable within the elongated sleeve when positioned therein, and wherein any sliding of the plunger inside the sleeve does not cause the movement of the elongated sleeve inside the cylindrical chamber of the barrel; a plunger rod, dimensioned for engaging the plunger and forcing the plunger slideably within the sleeve in the directions towards and away from the tip; wherein the sliding of the plunger towards the tip causes the fluid to be dispensed out the tip of the barrel and wherein the sliding of the plunger away from the tip causes the fluid to be drawn into the tip.

### BACKGROUND OF THE INVENTION

The present invention relates generally to syringes, and in particular, to a multipart syringe that is utilizes a coordinated sleeve and plunger for drawing fluids into or dispensing fluids out of the barrel.

The number of syringe designs are numerous, with the objectives of such designs being quite varied. Along with meeting stringent medical and governmental specifications and/or regulations, such constructions are (at least in some cases) conceived of with an eye towards construction simplicity and minimization of manufacturing cost. One example of a syringe design that specifies one or more of the foregoing objectives is described in U.S. Pat. No. 5,413,563.

Generally speaking, such prior art constructions comprise a cylindrical syringe barrel, a hypodermic needle coupled thereto, and a plunger within the syringe barrel which, when a force is exerted axially by an operator, creates a suction force drawing body fluids into the barrel and/or delivers fluid through the hypodermic needle. The purpose of the plunger is to provide an airtight seal between itself and the syringe barrel so that movement of the plunger up and down the barrel will allow liquid, blood or other fluids to be drawn into or forced out of the syringe through the distal end.

Typical syringes used for such purposes include glass syringes, in which the cylindrical barrel is made of glass and the plunger is a ground glass rod that closely fits within the cylindrical barrel. In order to eliminate leakage and at the same time reduce resistance to an acceptable level, close tolerances are necessary between the barrel and the plunger. However, such glass syringes suffer from several disadvantages, namely, they are expensive to manufacture since they require close tolerances, they cannot be easily mass-produced since the plungers often cannot be interchanged with one another and have to be individually fit with the barrel during the grinding process by the manufacturer; and they are susceptible to breakage.

In an attempt to overcome the foregoing, the prior art has recognized the use of plastic barrels with plastic or elastomeric plungers, a brief discussion of which can be found in the aforementioned U.S. Pat. No. 5,413,563. One of the difficulties apparently recognized however was the ability to provide a satisfactory quality and strength of the seal between the plunger and barrel. For example, a delicate balance is required between the achieving of a satisfactory leak-proof seal and the resulting difficulty in moving the plunger within the barrel.

It is thus believed that further improvements in syringe constructions are desired. In particular, it would be desirable to provide a syringe construction that had the benefits of glass components where close tolerances are needed, while simultaneously takes advantage of the use of plastic where less exacting tolerances and/or molded pieces can be employed.

### SUMMARY AND OBJECTS OF THE INVENTION

It is therefore an object of the present invention to provide an improved syringe construction that overcomes the foregoing perceived deficiencies.

It is another object of the present invention to provide an improved syringe construction that meets and/or exceeds all medical and governmental specifications and/or regulations.

An additional object is to provide an improved syringe construction that achieves all the advantages provided by a free-sliding glass syringe but which is significantly less costly to manufacture.

Yet another object of the present invention is to provide an improved syringe construction that meets all of the foregoing objectives and that can be easily mass-produced.

Still another object of the present invention is to provide an improved syringe construction that meets all of the foregoing objectives and that is not as susceptible to breakage as prior art constructions.

Still other objects and advantages of the invention will in part be obvious and will in part be apparent from the specification.

The invention accordingly comprises the features of construction, combination of elements and arrangement of parts and sequence of steps which will be exemplified in the construction, illustration and description hereinafter set forth, and the scope of the invention will be indicated in the claims.

Therefore, and generally speaking, the present invention is directed to a syringe for at least one of drawing a fluid into the syringe and dispensing a fluid substance therefrom, the syringe comprising a barrel comprising an inner surface defining a cylindrical chamber for retaining a dispensable fluid therein, a distal end terminating in a tip to which a needle is coupleable, and an open proximal end; an elongated sleeve positionable within the cylindrical chamber, the sleeve having an outer diameter dimensioned to be fittable in the cylindrical chamber; a plunger having a distal end and a proximal end, at least one of which is capped, the plunger being dimensioned to be slideable within the elongated sleeve when positioned therein, and wherein any sliding of the plunger inside the sleeve does not cause the movement of the elongated sleeve inside the cylindrical chamber of the barrel; a plunger rod, dimensioned for engaging the plunger and forcing the plunger slideably within the sleeve in the directions towards and away from the tip; wherein the sliding of the plunger towards the tip causes the fluid to be dispensed out the tip of the barrel and wherein the sliding of the plunger away from the tip causes the fluid to be drawn into the tip.

### BRIEF DESCRIPTION OF THE DRAWINGS

For a fuller understanding of the invention, reference is had to the following description taken in connection with the accompanying figures, in which:

FIG. 1 is a perspective view of a syringe constructed in accordance with the present invention;

FIG. 2 is a perspective view of the syringe of FIG. 1 from a different angle of viewing;

FIG. 3 is a perspective view of the components that comprise the syringe illustrated in FIGS. 1 and 2; and

FIG. 4 is a cross-sectional view of the syringe illustrated in FIGS. 1 and 2 in an assembled and operating position.

While all features may not be labeled in each Figure, all elements with like reference numerals refer to similar or identical parts.

### DETAILED DESCRIPTION OF THE PREFERRED EMBODIMENTS

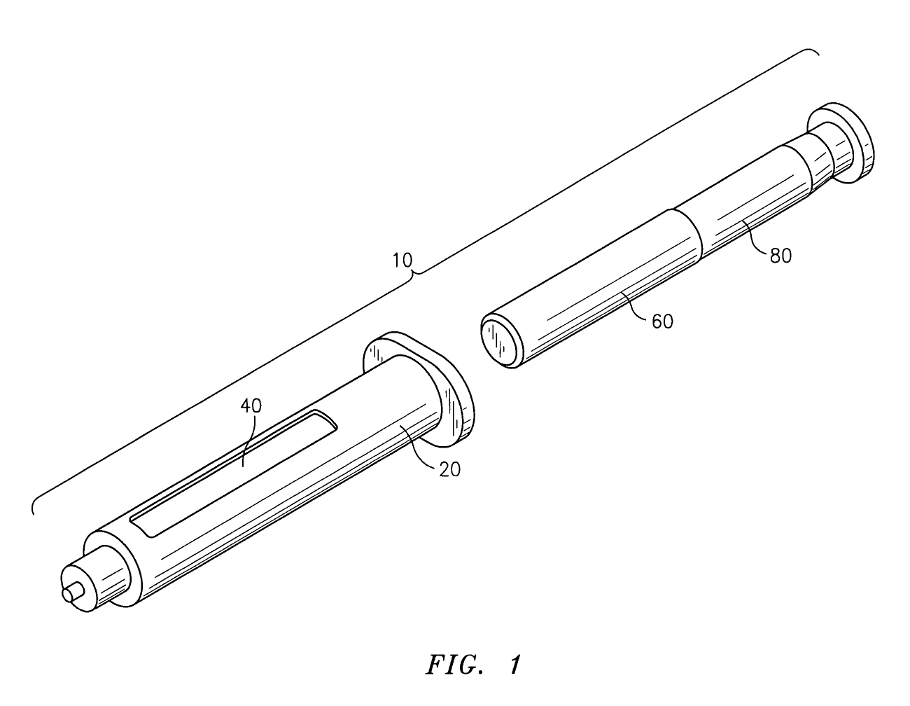

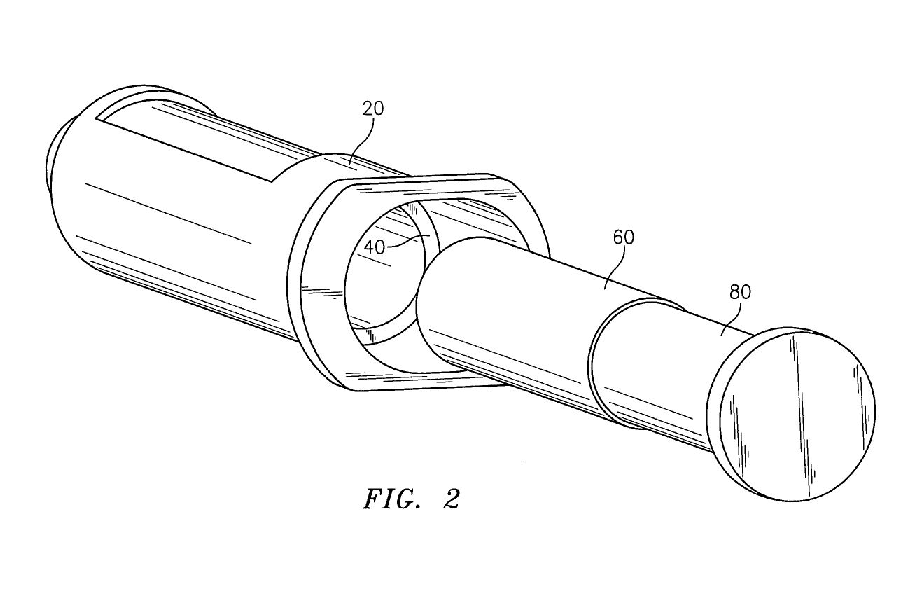

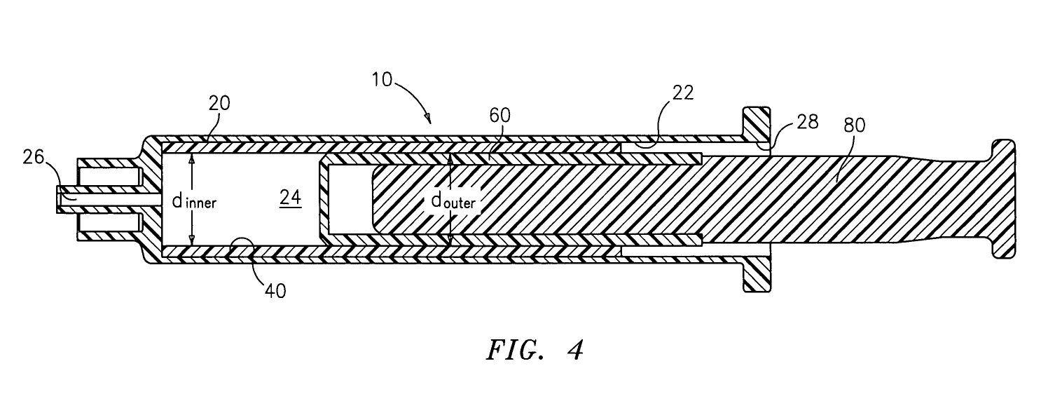

Reference will now be made to FIGS. 1-4, wherein a syringe, generally indicated at 10 and constructed in accordance with the present invention, is depicted.

Syringe 10, in its fully completed and operational configuration, is preferably constructed to at least one of draw in and/or dispense a fluid substance, such as liquid, blood, medicines (all collectively and individually hereinafter referred to generically as a “fluid”. Generally speaking, syringe 10 is comprised of four main components, namely a barrel generally indicated at 20, an elongated sleeve generally indicated at 40, a plunger generally indicated at 60, and a plunger rod generally indicated at 80. Reference to the particular construction and features of each of the foregoing components, as well as their interrelationship, will now be made.

Specifically, barrel 20 comprises an inner surface 22 defining a cylindrical chamber 24 for retaining a dispensable or injectable (collectively (“dispensable”)) fluid therein, a distal end terminating in a tip 26 to which a needle (not shown) is coupleable, and an open proximal end 28.

Elongated sleeve 40 is positionable within cylindrical chamber 24 and has an outer diameter dimensioned to be preferably friction-fittable in the cylindrical chamber. In the preferred embodiment, a suitable adhesive (the type/brand of which would be well known in the medical field) is used to ensure that once positioned inside chamber 24, sleeve 40 is not able to inadvertently or undesirably move. Preferably, the length of elongated sleeve 40 and plunger is about ⅔ of the length of barrel 20.

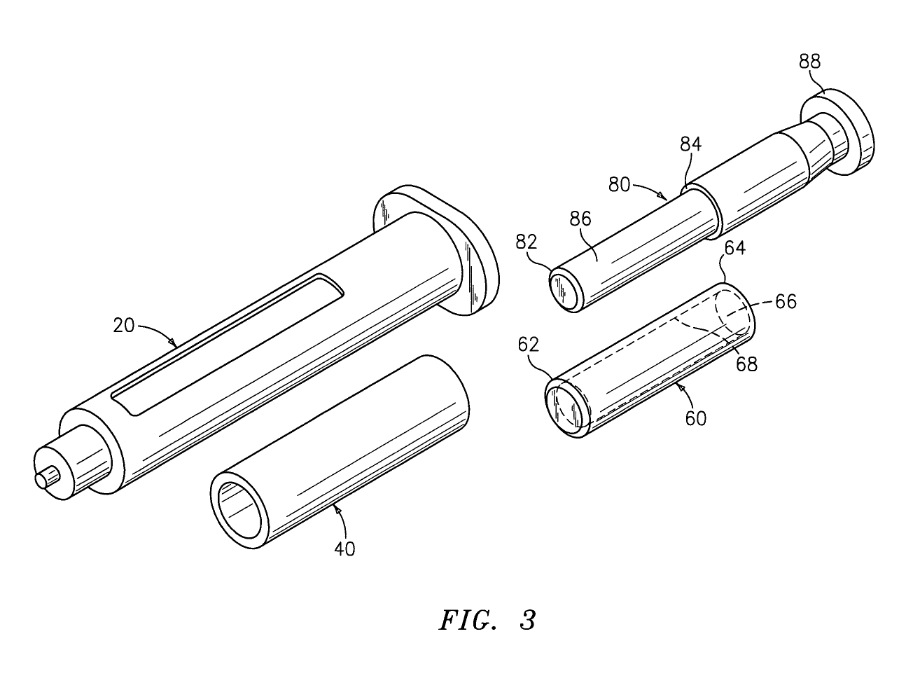

Plunger 60 has a distal end 62 and a proximal end 64, at least one of which is preferably (but not necessarily) capped. In the preferred embodiment (and as most easily illustrated in FIG. 3), it is distal end 62 that is capped. Plunger 60 is dimensioned to be slideable within sleeve 40 when positioned therein. In the preferred embodiment, there is about (and preferably no more than) a seven (7) micron gap between the inner diameter dᵢₙₙₑᵣ of sleeve 40 and the outer diameter dₒᵤₜₑᵣ of plunger 60 (see FIG. 3). This is an acceptable balance to provide the necessary suction and or pushing force while maintaining a relatively leak-free syringe. Importantly, any sliding of plunger 60 inside sleeve 40 does not cause the movement of sleeve 40 inside cylindrical chamber 24 of barrel 20.

The fourth of the main components comprising syringe 10 is plunger rod 80, which is dimensioned for engaging plunger 60 and forcing plunger 60 slideably within sleeve 40 towards (for dispensing) and away from (aspiration) tip 26. In this way, the sliding of plunger 60 towards tip 26 causes the fluid to be dispensed out tip 26 of barrel 20 and wherein the sliding of plunger 60 away from tip 26 causes the fluid to be drawn into tip 26.

As indicated above, in the preferred embodiment, distal end 62 of plunger 60 is the capped end and proximate end 64 of plunger 60 is open such that a distal end 82 of plunger rod 80 can be received in the cavity 66 formed in plunger 60. In this way, plunger rod 80 can force plunger 60 slideably within sleeve 40 and towards tip 26 by engaging the capped end from the cavity side of plunger 60. Alternatively and/or in addition, plunger rod 80 may comprise a shoulder 84 for engaging proximate end 64 (i.e. the lip) of plunger 60. In this way, shoulder 84 will assist in being able to force plunger 60 slideably within sleeve 40 and towards tip 26. Still further, a mere adhesive coupling and/or sufficient friction fitting of the outer surface 86 of plunger rod 80 and the inner surface 68 of plunger 60 may be sufficient to force plunger rod 60 towards and away from tip 26 by the respective pushing or pulling force upon an end (e.g. knob member) 88 of plunger rod 80.

As set forth above, the present invention achieves the objectives of meeting and/or exceeding all medical and governmental specifications and regulations, providing a lower cost construction and a practical means for mass production, and reducing undesirable breakage by preferably forming the barrel and plunger rod out of polypropylene (i.e. plastic), while providing the sleeve and plunger out of borosilicate glass (e.g. “glass”). It should be understood that the foregoing plastic and glass combination is only a preferred construction, as other materials meeting the desired functional and regulatory constraints could also be used while remaining within the scope of the present invention.

The operation of syringe 10 of the present invention is as follows. In preparation for use, a hypodermic or other suitable needle assembly is coupled (e.g. snapped on) to the tip end of barrel 20, with sleeve 40 already having been positioned and adhered to the inside surface of chamber 24. The combination plunger 60 and plunger rod 80 may then be slidably inserted into the inside cavity of sleeve 40. Aspiration and/or dispensing of the liquid can then take place in a conventional manner, with the practitioner essentially oblivious to the particular materials that comprise syringe 10.

It will be appreciated from the foregoing description that the syringe of the instant invention possesses all the attributes of a syringe comprised of all glass, while also providing the advantages provided by a syringe using plastic or other less expensive components. In the preferred embodiment, the plunger and sleeve are comprised of the same material (e.g. glass) to ensure the proper seal and sliding therebetween.

It will thus be seen that the objects set forth above, among those made apparent from the preceding description, are efficiently attained and, since certain changes may be made in the above constructions and methodologies without departing from the spirit and scope of the invention, it is intended that all matter contained in the above description or shown in the accompanying drawings shall be interpreted as illustrative and not in a limiting sense.

For example, the capped end may be provided at the proximate end of plunger 60, although this is less than optimal for securing the plunger rod thereto. Additionally, barrel 20 may be provided with a window though which the practitioner can see the amount of fluid in the barrel. Of course this latter feature forms no part of the invention.

It is also to be understood that the following claims are intended to cover all of the generic and specific features of the invention described herein and all statements of the scope of the invention that as a matter of language might fall there between.

What is claimed is:

1. A syringe for at least one of drawing a fluid into the syringe and dispensing a fluid substance therefrom, the syringe comprising:
a barrel comprising an inner surface defining a cylindrical chamber for retaining a dispensable fluid therein, a distal end terminating in a tip to which a needle is coupleable, and an open proximal end;
an elongated sleeve positionable within the cylindrical chamber, the sleeve having an outer diameter dimensioned to be fittable in the cylindrical chamber;
a plunger having a distal end and a proximal end, the plunger being dimensioned to be slideable within the elongated sleeve when positioned therein, and wherein any sliding of the plunger inside the sleeve does not cause the movement of the elongated sleeve inside the cylindrical chamber of the barrel;
a plunger rod, dimensioned for engaging the plunger and forcing the plunger slideably within the sleeve in the directions towards and away from the tip;
wherein the sliding of the plunger towards the tip causes the fluid to be dispensed out the tip of the barrel and/or wherein the sliding of the plunger away from the tip causes the fluid to be drawn into the tip.

2. The syringe as claimed in claim 1, wherein the distal end of the plunger is a capped end and the proximate end of the plunger is open such that a distal end of the plunger rod can be received in the cavity formed in the plunger, and
wherein the plunger rod forces the plunger slideably within the sleeve and towards the tip by engaging the capped end from the cavity side of the plunger.

3. The syringe as claimed in claim 1, wherein the distal end of the plunger is a capped end and the plunger rod comprises a shoulder, wherein the shoulder engages the proximate end of the plunger; and
wherein the plunger rod forces the plunger slideably within the sleeve and towards the tip by the engagement of the shoulder and the proximate end of the plunger.

4. The syringe as claimed in claim 1, wherein the barrel is made of polypropylene.

5. The syringe as claimed in claim 1, wherein the plunger rod is made of polypropylene.

6. The syringe as claimed in claim 1, wherein the sleeve is made of glass.

7. The syringe as claimed in claim 1, wherein the plunger is made of glass.

8. The syringe as claimed in claim 1, wherein at least the sleeve and the plunger are made of glass.

9. The syringe as claimed in claim 8, wherein the barrel and the plunger rod are made of polypropylene.
